(19) United States
(12) Patent Application Publication
(10) Pub. No.: US 2005/0147699 A1
(43) Pub. Date: Jul. 7, 2005

(54) Astragalus radix and codonopsis pilosulae radix mixed extract for inhibiting carcinogenesis and metastasis

(76) Inventors: Rey-Yuh Wu, Taipei (TW); Bor-Shiun Chen, Taipei (TW); Tzung-Hsien Lai, Taipei (TW); Yi-Peng Chen, Taipei (TW); Yuh-Shan Chung, Taipei (TW); Paw-Hwa Yang, Taipei (TW); Shu-Yin Su, Taipei (TW); Tsu-Hwie Liu, Taipei (TW)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/749,323

(22) Filed: Dec. 31, 2003

(51) Int. Cl. A61K 35/78

(52) U.S. Cl. 424/757; 424/773

(57) ABSTRACT

The present invention provides a composition for inhibiting carcinogenesis and metastasis, comprising a therapeutically effective amount of an Astragalus radix and Codonopsis pilosulae radix mixed extract. Methods of producing said Astragalus radix and Codonopsis pilosulae radix mixed extract are also provided.

### BACKGROUND OF THE INVENTION

1. Field of the invention

The invention mainly relates to a herbal extract; more particularly, to an Astragalus radix and Codonopsis pilosulae radix mixed extract having the ability to inhibit carcinogenesis and metastasis.

2. Description of the Related Art

Astragalus radix (Astragali mogholici radix or Astragali membranacei radix), known as Huang-chi or Huang Qi, and Codonopsis pilosulae radix (Codonopsis pilosulae radix, Codonopsis tangshen radix or Codonopsis modestae radix), known as Dangshen, are well known basic drugs in traditional Chinese medicine. They have been formulated with other herbs for use as tonics, diuretics or anti-perspirants for thousands of years.

Huang-chi is the dried root of Astragalus mogholicus or Astragalus membranaceus, particularly Astragalus membranaceus Bge. var. mongholicus (Bge.) Hsiao or A. membranaceus (Fisch.) Bge. (Leguminosae). It is recorded to treat chronic nephritis, albuminuria, myositis, antihypertensive, coronary artery disease, cerebral infarction, peptic ulcer (duodenal and gastric ulcer), renal disease and diabetes mellitus, in the traditional pharmacopoeia. In addition, several Huang-chi extracts can be used to treat tumors. Some studies suggest that the mechanism of treating tumors is through modulating immune responses (Lau, B. H. S., Ruckle, H. C., Botolazzo, T. and Lui, P. D. Chinese medicinal herbs inhibit growth of murine renal cell carcinoma. Cancer Biotherapy. Vol. 9, No. 24, pp. 153-161. 1994; Rittenhouse, J. R., Lui, P. and Lau, B. H. S. Chinese medicinal herbs reverse macrophage suppression induced by urological tumors. The Journal of Urology. Vol. 146, pp. 486-490, 1991), while some studies demonstrate that the mechanism is through modulating mutagenesis (Wong, B. Y. Y., Lau, B. H. S., Tadi, P. P. and Teel, R. W. Chinese medicinal herbs modulate mutagenesis, DNA binding and metabolism of aflatoxin B₁. Mutation Research. Vol. 279, pp. 209-216, 1992). Rittenhouse et al, 1991, further discloses that Astragalus membranaceus increases phagocytosis of macrophage.

Recently, the contents of Huang-chi have been assayed to comprise monosaccharides, polysaccharides, flavones, amino acids, and microelements. Among them, polysaccharides are emphasized on their rich content. Furthermore, polysaccharides have been proved to have pharmaceutical effects on treating diseases, especially immune-related diseases, such as tumors (U.S. Pat. No. 5,268,467; Tang, W., Hemm, I. And Bertram, B. Recent development of antitumor agents from Chinese herbal medicines, Part II. High molecular compounds. Planta Med. Vol. 69, pp. 193-201, 2002; EP Application No. 91101424.9 and Japan Publication No. 1993-39305; PCT International Publication No. WO 94/04162), bacterial and viral infections (EP Application No. 91101424.9; Japan Publication No. 1993-39305; PCT International Publication No. WO 94/04162), and asthmatic airway inflammation (Xue, J., Xu, Y., Zhang, Z., Shen, G. and Zeng G. The effect of Astragapolysaccharide on the lymphocyte proliferation and airway inflammation in sensitized mice. Journal of Tongji Medical University, Vol. 19, No. 1, pp. 20-22, 1999).

Yoshida et al (Yoshida, Y., Wang, M. Q., Liu, J. N., Shan, B. E. and Yamashita, U. Immunomodulating activity of Chinese medicinal herbs and Oldenlandia diffusa in particular. Int. J Immunopharmac., Vol 19, No. 7, pp. 359-370, 1997) further evidence that Astragalus membranaceus markedly simulate murine spleen cells to proliferate. They also demonstrate that A. membranaceus increases the total amount of T cells and helper T cells and also stimulates T cell proliferation. Furthermore, A. membranaceus is evidenced to modulate immune response in a concentration-dependent manner, of which low concentration is to enhance the induction of T lymphocyte, while high concentration is to inhibit. In the aspect of B cells, administration of A. membranaceus stimulates immunoglobulin G production and helps immune complexes cleaning. In addition, Chen discloses that Huang-chi can stimulate IL-2 production by spleen lymphocytes (Chen, Y. C. Experimental studies on the effects of danggui buxue decoction on IL-2 production of blood-deficient mice. Zhongguo Zhong Yao Za Zhi. Vol. 19, No. 12, pp. 739-741, 1994).

Besides the immune system, polysaccharides from Huang-chi were also found to have effect on the reticuloendothelial system (Shimizu, N., Tomoda, M., Kanary, M and Gonda, R. An acidic polysaccharide having activity on the reticuloendothelial system from the root of Astragalus mongholicus. Chem. Pharm. Bull. Vol. 39, No. 11, pp. 2969-2972, 1991). An et al, PCT International Publication No. WO 01/00682, discloses purified arabinogalactan compositions isolated from Astragalus membranaceus and arabinogalactan protein compositions having an average molecular weight of at least 100 kiloDalton isolated from these purified arabinogalactan compositions are useful for stimulating hematopoiesis, inducing the proliferation or maturation of megakaryocytes, stimulating the production of IL-1β, IL-6, TNF-α, TFN-γ, GM-CSF, or G-CSF, stimulating the production or action of neutrophils, treating neutropenia, anemia, or thrombocytopenia, accelerating recovery from exposure to cytotoxic agents or radiation, treating cachexia, emesis, or drug withdrawal symptoms, or modifying biological responses or protecting hepatic cells in hepatitis B.

A series of polysaccharides such as astragalans I-III, AG-I, AG-II, AH-I, AH-II, Amem-P, and Amon-S have been isolated from the roots of Astragalus membranaceus or Astragalus membranaceus var. mongholicus, recently (Tang et al; 2003).

Dangshen is the dried root of Codonopsis pilola (Franch.) Nannf, Codonopsis tangshen Oliv. or Codonopsis pilola (Franch.) var. modesta (Nannf.) L. T. Shen that belongs to the family, Campanulaceae. It has been employed for the treatment of dyspepsia, poor appetite, fatigue, peptic ulcer, damage of gastric mucosa, nephrogenic anemia and psychoneurosis, and is sometimes used as a substitute of the much more costly Panax ginseng. Recently, the contents of Dangshen have been assayed to mainly comprise polysaccharides and saponins. The polysaccharides of Dangshen are proved to have the ability to treat and prevent cardiovascular disease (U.S. Pat. No. 4,999,343). As to immunomodulatory effect, the polysaccharides of Dangshen lowered mitogenic response to Concanavalin A (ConA) and lipopolysaccharide of splenocytes and weakly stimulate human lymphocytes to proliferate (Wang, Z. T., Ng, T. B., Yeung, H. W., and Xu, G. J. Immunomodulatory effect of a polysaccharide-enriched preparation of Conconopsis polisula roots. Gen. Pharmac. Vol. 27, No. 8, pp. 1347-1350, 1996; Shan, B. E., Yoshida, Y., Sugiura, T., and Yamashita, U. Stimulating activity of Chinese medicinal herbs on human lymphocytes in vitro. International Journal of Immunopharmacology. Vol. 21, pp. 149-159, 1999). Dangshen also has effect on improving survival in systemic lupus erythematosus patients by improving defective interleukin-2 production (Chen, J.-R., Yen, J.-H., Lin, C.-C., Tsai, W.-J., Liu, W.-J., Tsai, J.-J., Lin, S.-F., and Liu, H.-W. The effects of Chinese herbs on improving survival and inhibiting anti-ds DNA antibody production in lupus mice. American Journal of Chinese Medicine. Vol. XXI. Nos. 3-4, pp. 257-262, 1993). A series of water-soluble polysaccharides such as CP-1, CP-2, CP-3, and CP-4 have been isolated from the roots of C. polisula (Zhang, S. J. and Zhang, S. T. Studies of polysaccharides of Codonopsis pilosula. Zhong Zao Yao. Vol. 18, No. 8, pp. 2-4, 1987).

Compositions containing Huang-chi and/or Dangshen are developed (China Patent Application Nos. No. 93110434.3, 94104243.X, 94101734.6, and 96117419.6, U.S. Pat. Nos. 4,618,495 and 4,843,067). For example, Okuda et al, U.S. Pat. No. 4,618,495, disclose a composition for reducing cancer symptoms by improving lipid metabolism and eliminating or reducing anorexia in tumor-bearing patients through inhibition of the lipid degradation-promoting action of toxohormone L. The composition comprises an aqueous or aqueous organic solvent extract of one or more crude preparations selected from the group consisting of Astragali radix (Huang-chi), Cinnamomi cortex, Rehmanniae radix, Paeoniae radix, Cnidii rhizoma, Astractylodis lanceae rhizoma, Angelicae radix, Ginseng radix, Hoelen and Glycyrrhizae radix.

However, there are no prior arts that teach or imply use of Huang-chi and Dangshen for treating tumors.

### SUMMARY OF THE INVENTION

The invention provides a composition for inhibiting carcinogenesis and metastasis, comprising a therapeutically effective amount of an Astragalus radix and Codonopsis pilosulae radix mixed extract. Preferably, the weight ratio of Astragalus radix:Codonopsis pilosulae radix in the mixed extract is from 3:1 to 1:3.

According to the invention, the Astragalus radix and Codonopsis pilosulae radix mixed extract is unexpectedly found to have dramatic effects on inhibiting carcinogenesis and metastasis. Preferably, the mixed extract according to the invention is directed to colon cancer, lung carcinoma and/or mammary adenocarcinoma.

### BRIEF DESCRIPTION OF THE DRAWINGS

FIG. 1 illustrates the GPC spectrograms of the Astragalus radix and Codonopsis pilosulae radix mixed extract in Example 3; a: DSWH>10 K; b: DSWH 5-10 K; c: DSWH,5K; and d: HG-021024-UF>10 K.

FIG. 2 illustrates the GPC spectrograms of the Astragalus radix extract and Codonopsis pilosulae radix extract in Example 4; a: polysaccharides from Astragalus radix before hollow fiber filtration; b: polysaccharides from Astragalus radix after hollow fiber filtration; c: polysaccharides from Codonopsis pilosulae before hollow fiber filtration; and d: polysaccharides from Codonopsis pilosulae after hollow fiber filtration.

FIG. 3 illustrates the GPC spectrograms of the Astragalus radix extract and Codonopsis pilosulae radix extract in Example 5; a: polysaccharides from Codonopsis pilosulae; and b: polysaccharides from Astragalus radix.

FIG. 4 illustrates the GPC spectrograms of the Astragalus radix extract and Codonopsis pilosulae radix extract prepared in Example 5; a: standard solutions; b: DS-3; c: DS>50 k; and d: HG>50 k.

FIG. 5 illustrates the nodule number in the liver of BALB/c mice after intra-splenic implantation with colon cancer cell CT-26; NC: without treatment; SG-W-1.7: treated with 1.7 g/kg of SG-W; SG-W-0.6: treated with 0.6 g/kg of SG-W; SG-F1-0.6: treated with 0.6 g/kg of SG-F1; SG-F1-0.2: treated with 0.2 g/kg of SG-F1; SG-F2-0.6: treated with 0.6 g/kg of SG-F2; SG-F2-0.2: treated with 0.2 g/kg of SG-F2; SG-F3-0.6: treated with 0.6 g/kg of SG-F3; and SG-F3-0.2: treated with 0.2 g/kg of SG-F3; *: p<0.05 when compared with NC; **: p<0.01 when compared with NC.

FIG. 6 illustrates the nodule number in the liver of BALB/c mice after intra-splenic implantation with colon cancer cell CT-26; NC: without treatment; SG-F3-0.02: treated with 0.02 g/kg of SG-F3; SG-F3-0.05: treated with 0.05 g/kg of SG-F3; SG-F3-0.1: treated with 0.1 g/kg of SG-F3; SG-F3-0.2: treated with 0.2 g/kg of SG-F3; SG-F3-0.6: treated with 0.6 g/kg of SG-F3; *: p<0.05 when compared with NC.

FIG. 7 illustrates the nodule number in the liver of BALB/c mice after intra-splenic implantation with colon cancer cell CT-26 and treated with the extracts prepared in Example 3; NC: without treatment; DS-0.6: treated with 0.6 g/kg of the Codonopsis pilosulae radix extract; DS-0.3+HG-0.3: treated with 0.3 g/kg of the Codonopsis pilosulae radix extract and 0.3 g/kg of Astragalus radix extract; HG-0.6: treated with Astragalus radix extract; **: p<0.01 when compared with NC.

FIG. 8 illustrates the nodule number in the liver of BALB/c mice after intra-splenic implantation with colon cancer cell CT-26 and treated with the extracts prepared in Example 4; NC: without treatment; SG-10: treated with 0.6 g/kg of the Codonopsis pilosulae radix extract; SG-91: treated with the Codonopsis pilosulae radix extract and the Astragalus radix extract in a ratio of 9:1; SG-31: treated with the Codonopsis pilosulae radix extract and the Astragalus radix extract in a ratio of 3:1; SG-11: treated with the Codonopsis pilosulae radix extract and the Astragalus radix extract in a ratio of 1:1; SG-13: treated with the Codonopsis pilosulae radix extract and the Astragalus radix extract in a ratio of 1:3; SG-19: treated with the Codonopsis pilosulae radix extract and the Astragalus radix extract in a ratio of 1:9; SG-01: treated with 0.6 g/kg of the Astragalus radix extract; *: p<0.05 when compared with NC.

FIG. 9 illustrates the nodule number in the liver of BALB/c mice after intra-splenic implantation with colon cancer cell CT-26 and treated with the extracts prepared in Example 5; NC: the negative control group of mice were conducted intra-splenic implantation of mouse colorectal cancer CT-26 cells and were administered with Mili-Q water by intragastric gavage daily for 21 consecutive days; Sham: the sham group of mice were the same as the NC group of mice except that the mice were injected with the sterile normal saline to substitute for CT-26 cell suspension; SGCF-10: treated with 0.6 g/kg of the Codonopsis pilosulae radix extract; SGCF-91: treated with the Codonopsis pilosulae radix extract and the Astragalus radix extract in a ratio of 9:1; SGCF-31: treated with the Codonopsis pilosulae radix extract and the Astragalus radix extract in a ratio of 3:1; SGCF-11: treated with the Codonopsis pilosulae radix extract and the Astragalus radix extract in a ratio of 1:1; SGCF-13: treated with the Codonopsis pilosulae radix extract and the Astragalus radix extract in a ratio of 1:3; SGCF-19: treated with the Codonopsis pilosulae radix extract and the Astragalus radix extract in a ratio of 1:9; SGCF-01: treated with 0.6 g/kg of the Astragalus radix extract; *: p<0.05 when compared with NC.

FIG. 10 illustrates tumor weight of the JC cell line implanted in the subdermal area of BALB/c mice; NC: without treatment; SG-F3-0.1: treated with 0.1 g/kg of SG-F3; SG-F3-0.3: treated with 0.3 g/kg of SG-F3; SG-F3-0.6: treated with 0.6 g/kg of SG-F3; *: p<0.05 when compared with Co; **: p<0.01 when compared with Co.

FIG. 11 illustrates tumor weight of the JC cell line implanted in the subdermal area of BALB/c mice treated with the extracts prepared in Example 5; NC: without treatment; G: treated with 0.2 g/kg of the Astragalus radix extract; S:G 1 1:9: treated with the Codonopsis pilosulae radix extract and the Astragalus radix extract in a ratio of 1:9; S:G2 1:3: treated with the Codonopsis pilosulae radix extract and the Astragalus radix extract in a ratio of 1:3; S:G 3 1:1: treated with the Codonopsis pilosulae radix extract the Astragalus radix extract in a ratio of 1:1; S:G 4 3:1: treated with the Codonopsis pilosulae radix extract and the Astragalus radix extract in a ratio of 3:1; S:G 5-9:1: treated with the Codonopsis pilosulae radix extract and the Astragalus radix extract in a ratio of 9:1; S: treated with 0.2 g/kg of the Codonopsis pilosulae radix extract; *: p<0.05 when compared with Con.

FIG. 12 illustrates tumor weight of the LL/2 cell line implanted in the subdermal area of BALB/c mice treated with the extracts prepared in Example 5; NC: without treatment; S: treated with 0.2 g/kg of the Codonopsis pilosulae radix extract; S:G 1 1:9: treated with the Codonopsis pilosulae radix extract and the Astragalus radix extract in a ratio of 1:9; S:G 2 1:3: treated with the Codonopsis pilosulae radix extract and the Astragalus radix extract in a ratio of 1:3; S:G 3 1:1: treated with the Codonopsis pilosulae radix extract and the Astragalus radix extract in a ratio of 1:1; S:G 4 3:1: treated with the Codonopsis pilosulae radix extract and the Astragalus radix extract in a ratio of 3:1; S:G 5-9:1: treated with the Codonopsis pilosulae radix extract and the Astragalus radix extract in a ratio of 9:1; G: treated with 0.2 g/kg of the Astragalus radix extract; *: p<0.05 when compared with Con.

### DETAILED DESCRIPTION OF THE INVENTION

The present invention mainly provides a composition for inhibiting carcinogenesis and metastasis comprising a therapeutically effective amount of a Astragalus radix and Codonopsis pilosulae radix mixed extract. The effect of the composition according to the invention is better than that of Astragalus radix or Codonopsis pilosulae radix solely.

According to the invention, the ratio of Astragalus radix and Codonopsis pilosulae radix depends on the tumor to be treated. In one preferred embodiment of the invention, the weight ratio of Astragalus radix:Codonopsis pilosulae radix in the mixed extract is from 9:1 to 1:9. In one more preferred embodiment of the invention, the weight ratio is from 3:1 to 1:3. In the most preferred embodiment of the invention, the weight ratio is 3:1 or 1:1.

According to the invention, Astragalus radix is Astragalus mogholicus or Astragalus membranaceus radix. For example, Astragalus radix is Astragalus membranaceus Bge. var. mongholicus (Bge.) Hsiao or A. membranaceus (Fisch.) Bge. (Leguminosae).

According to the invention, Codonopsis pilosulae radix is Codonopsis pilosula radix. For example, Codonopsis pilosulae radix is Codonopsis pilosula (French.) Nannf., Codonopsis pilosula (French.) Nannf. var. modesta (Nannf.) L. T. Shen. or Codonopsis tangshen Oliv.

The composition according to the invention has the ability to inhibit carcinogenesis and metastasis. Preferably, the composition is effective on solid tumors, and wherein more preferably, the composition is for inhibiting carcinogenesis and metastasis of colon cancer, lung carcinoma or mammary adenocarcinoma. In the animal model illustrated in the invention, the composition according to the invention is for use in treating hepatic nodule metastasis from colon cancer and in treating mammary adenocarcinoma and lung carcinoma implanted in subdermal area. The weight of the tumor is observed to be significantly reduced after administrating the composition according to the invention comparing with the untreated control group or treated with Astragalus radix extract or Codonopsis pilosulae radix extract only.

According to the invention, the composition is administrated in several forms. It may be consumed as a pharmaceutical composition or as a food composition. Preferably, the composition according to the invention is in the form of tablet, capsule, solution, tonic, or food.

The Astragalus radix and Codonopsis pilosulae radix mixed extract contained in the composition according to the invention can be produced by many methods. The Astragalus radix and Codonopsis pilosulae radix mixed extract may be co-extracted or extracted solely and then combined together to obtain the mixed extract. Conventional methods of producing herb extract can be applied in the invention.

In one embodiment of the invention, a method of producing the Astragalus radix and Codonopsis pilosulae radix mixed extract according to the invention comprises (A) co-extracting Astragalus radix and Codonopsis pilosulae radix with water. More particularly, the method comprises:
(A11) co-extracting Astragalus radix and Codonopsis pilosulae radix with water at the temperature of from 45 to 95° C., preferably from 65 to 85° C., to separate a water-insoluble fraction from a water-soluble fraction; and
(A12) concentrating the water-soluble fraction at the temperature of from 40 to 80° C., preferably from 50 to 60° C., and at the pressure of from 0 to 100 mmHg.

In another aspect, the duration of (A11) co-extracting Astragalus radix and Codonopsis pilosulae radix with water is from 1 to 4 hours, and wherein preferably is from 1 to 2 hours. The co-extracting may be performed for several times; preferably for 1 to 4 times, and wherein more preferably for 2 to 3 times. The water-insoluble fraction separated in the former co-extraction is subjected to the subsequent co-extraction, and the water-soluble fractions are collected.

Preferably, the water-insoluble fraction is separated from the water-soluble fraction by filtration. For example, a filter of #100 to #400 mesh, preferably a filter of #325 to #400 mesh, is used.

In another embodiment of the invention, a method of producing the Astragalus radix and Codonopsis pilosulae radix mixed extract according to the invention comprises (B) co-extracting Astragalus radix and Codonopsis pilosulae radix with water and ethanol. More particularly, the method comprises:
(B1) co-extracting Astragalus radix and Codonopsis pilosulae radix with 90 to 99%, preferably 95%, ethanol to separate an ethanol-insoluble fraction from an ethanol-soluble fraction;
(B2) extracting the ethanol-insoluble fraction from step (B1) with water at the temperature of from 45 to 95° C. and obtaining a water-soluble fraction; and
(B3) combining the ethanol-soluble fraction from step (B1) and the water-soluble fraction from step (B2) and concentrating at the temperature of from 40 to 80° C. and at the pressure of from 0 to 100 mmHg.

Co-extracting step (B1) is similar to co-extracting step (A11) as mentioned above, except for the use of ethanol instead of water. In order to raise yield, co-extracting step (B1) may be performed for several times.

Preferably, the ethanol in the ethanol-insoluble fraction is removed after step (B1) for following manipulations. The method of ethanol removal is well known by artisans skilled in the field. For example, the ethanol-insoluble fraction is air dried to remove residue ethanol.

In step (B2), the ethanol-insoluble fraction from step (B1) is further extracted with water, which is also similar to co-extracting step (A11).

In step (B3), the ethanol-soluble fraction from step (B1) and the water-soluble fraction from step (B2) are combined and subjected to concentrating that is similar to step (A12). In addition, the ethanol-soluble fraction from step (B1) and the water-soluble fraction from step (B2) are optionally concentrated before combination.

Furthermore, the method optionally comprises precipitating the extract from step (B3) with ethanol. The supernatant and the pellet both have effects on inhibiting carcinogenesis and metastasis. The precipitation with ethanol is well known by persons skilled in the art.

In still another embodiment of the invention, a method of producing the Astragalus radix and Codonopsis pilosulae radix mixed extract according to the invention, comprises:
(A21) extracting Astragalus radix and Codonopsis pilosulae radix with water, respectively, to separate a water-insoluble from a water-soluble fraction;
(A22) ultra-filtrating the water-soluble fraction from step (A21) with an ultrafilter having a 5 to 10 kD molecular weight cutoff;
(A23) concentrating the filtrates from step (A22); and
(A24) combining and mixing filtrates of Astragalus radix and Codonopsis pilosulae radix from step (A23) to yield the Astragalus radix and Codonopsis pilosulae radix mixed extract.

The extracting step (A21) is similar to co-extracting step (A11) as mentioned above except Astragalus radix and Codonopsis pilosulae radix being extracted solely instead of co-extracted. In order to raise yield, the extracting step (A21) may be performed for several times.

According to the invention, step (A22) of ultra-filtrating the water-soluble fraction from step (A21) with an ultrafilter having a 5 to 10 kD molecular weight cutoff may be performed stepwisely. For example, ultra-filtrating the water-soluble fraction of Astragalus radix and Codonopsis pilosulae radix in step (A22) is with an ultrafilter having a 5 kD molecular weight cutoff and then with an ultrafilter having a 10 kD molecular weight cutoff. The filtrates from the 5 kD molecular weight cutoff ultrafilteration are concentrated and then subjected to the 10 kD molecular weight cutoff.

According to the invention, step (A23) of concentrating is similar to step (A12) as mentioned above.

Furthermore, step (A24) of combining, mixing and concentrating is also similar to step (B3).

In still another embodiment of the invention, a method of producing the Astragalus radix and Codonopsis pilosulae radix mixed extract according to the invention comprises:
(A31) extracting Astragalus radix and Codonopsis pilosulae radix with water, respectively, to separate a water-insoluble fraction from a water-soluble fraction;
(A32) concentrating the water-soluble fraction from step (A31) at the temperature of from 40 to 80° C. and at the pressure of from 0 to 100 mmHg;
(A33) ultra-filtrating and ultra-dialyzing the concentrated water-soluble fraction from step (A32) with an ultrafilter having a 1 to 4000 kD molecular weight cutoff;
(A34) mixing the filtrates from step (A33) to yield the Astragalus radix and Codonopsis pilosulae radix mixed extract.

The extracting steps (A31) and (A32) are similar to step (A21) and (A12), respectively, as mentioned above.

According to the invention, ultra-filtrating and ultra-dialyzing the concentrated water-soluble fraction from step (A32) of step (A33) is performed with a hollow fiber filtration cartridge and/or a cross-flow sluice cassette.

According to the invention, the hollow fiber filtration cartridge can remove insoluble residues and small molecular weight components. Preferably, a 0.2 μm hollow fiber is utilized. In addition, the cross-flow sluice cassette can remove small molecular weight components. Preferably, a membrane having 10 kD molecular weight cutoff is applied in the cross-flow sluice cassette. The hollow fiber filtration cartridge and the cross-flow sluice cassette can be applied solely or consequently.

Preferably, GPC system is applied for monitoring the molecular weight contribution of polysaccharides. When the area of the S phase is less than 5% of the total area, the ultra-filtration is completed.

In one embodiment of the invention, the molecular weight of Astragalus radix extract in the mixed extract is ranged from 10,000 to 1,000,000 D and the molecular weight of Codonopsis pilosulae radix extract in the mixed extract is ranged from 3,000 to 1,000,000 D based on the estimate of GPC analysis.

In the aspect of extracting efficiency, it ranges from 2 to 55% varying with the methods chosen.

Furthermore, the Astragalus radix and Codonopsis pilosulae radix mixed extract is dried as powder for the convenience of administration. The method of drying is well known to persons skilled in the art.

The following Examples are given for the purpose of illustration only and are not intended to limit the scope of the present invention.

### EXAMPLE 1

### Astragalus Radix and Codonopsis Pilosulae Radix Mixed Extract with Water (SG-W)

The ground Astragalus radix with a 190 g weight and 190 g of Codonopsis pilosulae radix were placed together in a 5 L tank. The ground herbs were stirred in 3.8 L of distilled water for 1 hour. The resulting opaque suspension was filtered with a #100 filter. The water-insoluble fraction was extracted again with 3.8 L of distilled water. The water-soluble fractions from the two steps of extraction were collected to provide 6.5 L of extract for concentrating under reduced pressure and dried. 197 g of brown power was prepared with a yield of 51.3%.

### EXAMPLE 2

### Astragalus Radix and Codonopsis Pilosulae Radix Mixed Extract with Ethanol and Water (SG-F1, SG-F2, and SG-F3)

The ground Astragalus radix with a 190 g weight and 190 g Codonopsis pilosulae radix were placed together in a 5 L tank. The ground herbs were stirred in 3.8 L of 95% ethanol for 1 hour. The resulting opaque suspension was filtered with a #100 filter. The ethanol-insoluble fraction was extracted again with 3.8 L of 95% ethanol. The ethanol-soluble fractions from the two steps of extraction were combined and then subjected to filtration, concentration under reduced pressure and dried. The resulting brown extract (SG-F1) was prepared.

In another aspect, the ethanol-insoluble fraction was air dried at 60° C. for 24 hours for removing ethanol and stirred with 5.7 L of distilled water for 1 hour. The water-insoluble fraction was extracted again with 3.8 L of distilled water. The water-soluble fractions from the two steps of extraction were collected and concentrated under reduced pressure to provide 760 mL of extract. The water-soluble fractions were added slowly with 3 L of 95% ethanol and mixed at 4° C. for 18 hours. The resulting supernatant was concentrated under reduced pressure and dried to obtain the extract (SG-F2). Additionally, the pellet was freeze-dried to obtain A light yellow power (SG-F3).

### EXAMPLE 3

### Astragalus Radix Extract and Codonopsis Pilosulae Radix Extract Prepared with Ultra-filtration

The 1.5 Kg ground Codonopsis pilosulae radix was stirred with 10× of distilled water twice and the 24 L of water-soluble fractions were combined. The resulting water-soluble fractions were ultra-filtrated with NF 2012 UF 10 K filter and AICRO™ Prefilter PP 5 u. Twelve L of water-soluble fraction was drained from a storage tank and filtered with Prefilter 5 u and UF 10 K and pumped by a Diaphragm Pump™ to obtain a filtrate (DSWH<10 K) and a concentrated solution (DSWH>10 K). During the ultra-filtration, distilled water was supplemented to maintain the total volume. The supplement was terminated when the filtrate appeared to have a very light yellow color. The 6.0 L concentrated solution (DSWH>10 K) was concentrated under reduced pressure to a volume of 0.5 L and dried at 60° C. overnight. The weight of the resulting brown polysaccharides was 19.1 g with a yield of 2.5%.

In another aspect, the filtrate (DSWH<10 K) was further filtered with Prefilter 5 u and UF 10 K and pumped by a Diaphragm Pump™ to obtain a filtrate (DSWH<5 K) and a concentrated solution (DSWH 5-10 K). When the volume of the concentrated solution (DSWH 5-10 K) was reduced to 12 L, it was subjected to concentration under reduced pressure to a volume of 0.7 L. The concentrated solution was then added with 2.1 L of ethanol and mixed for 2 hours. After centrifugation and freeze-dried, 25.1 g of ivory polysaccharides were obtained with a yield of 3.3%.

The 1.5 Kg ground Astragalus radix was stirred with 10× of distilled water twice and the 25 L of water-soluble fractions were combined. The resulting water-soluble fractions were ultra-filtrated with NF 2012 UF 10 K filter and AICRO™ Prefilter PP 5 u. The water-soluble fraction was drained from a storage tank and filtered with Prefilter 5 u and UF 10 K and pumped by a Diaphragm Pump™ to obtain a filtrate (HGWH<10 K) and a concentrated solution (HGWH>10 K). During the ultra-filtration, distilled water was supplemented to maintain the total volume. The supplement was terminated when the filtrate appeared to have a very light yellow color. The concentrated solution (HGWH>10 K) was concentrated under reduced pressure and dried at 60° C. overnight. The weight of the resulting brown polysaccharides (HG-021024-UF>10 K) was 95.1 g with a yield of 6.3%.

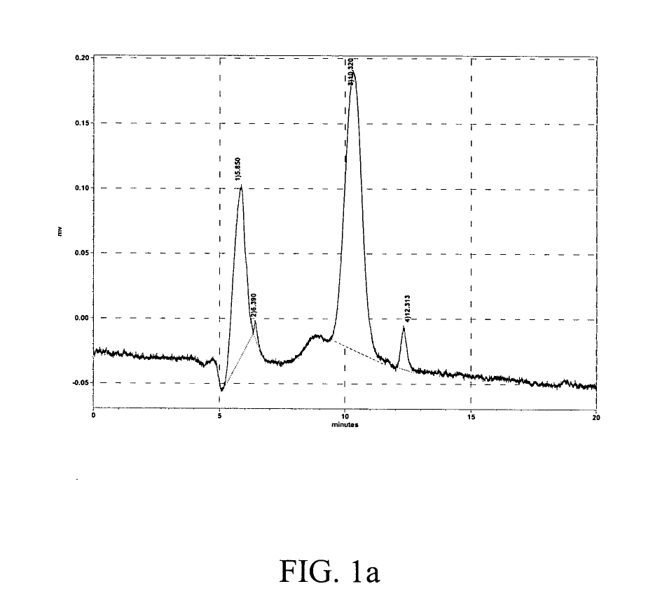

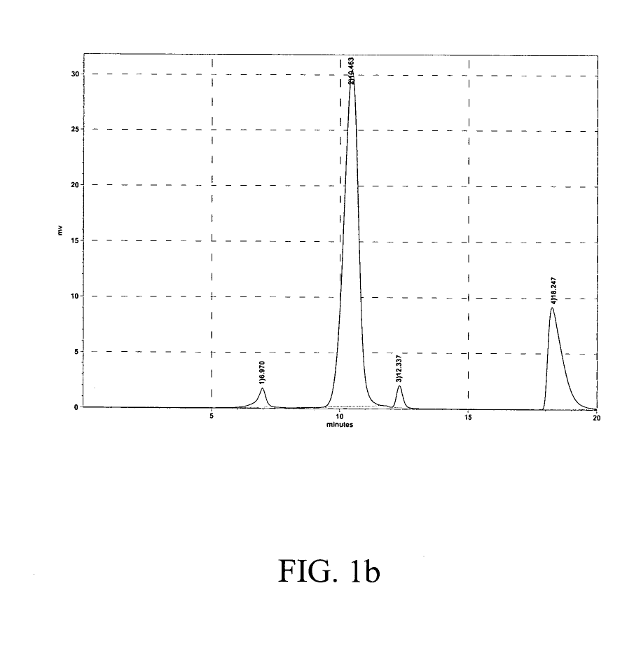

The Astragalus radix extract and Codonopsis pilosulae radix extract were analyzed with a GPC spectrometer. The spectrograms are shown in FIG. 1.

### EXAMPLE 4

### Astragalus Radix Extract and Codonopsis Pilosulae Radix Extract Prepared with Hollow Fiber Filtration Cartridge

The 50 Kg ground Codonopsis pilosulae radix was placed in a 1500 L tank for heating to 85° C. and stirring with 500 L of distilled water. The resulting opaque suspension was filtered with a #400 filter. The water-insoluble fraction was heated to 85° C. and stirred again with 360 L of distilled water for 2 hours and the insoluble fraction was removed with a #400 filter and centrifugation. The water-soluble fraction from the extractions was collected to provide 774 L of extract for concentrating under 76 cmHg at 55° C. The resulting brown extract with a 55.5 Kg weight was obtained.

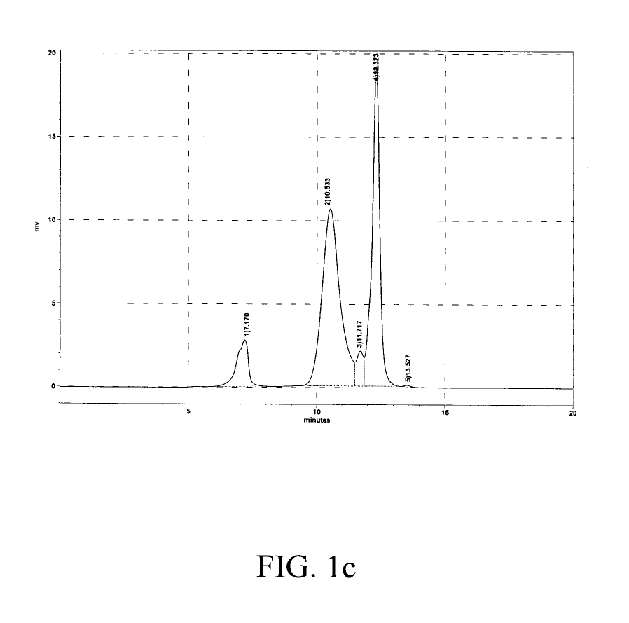

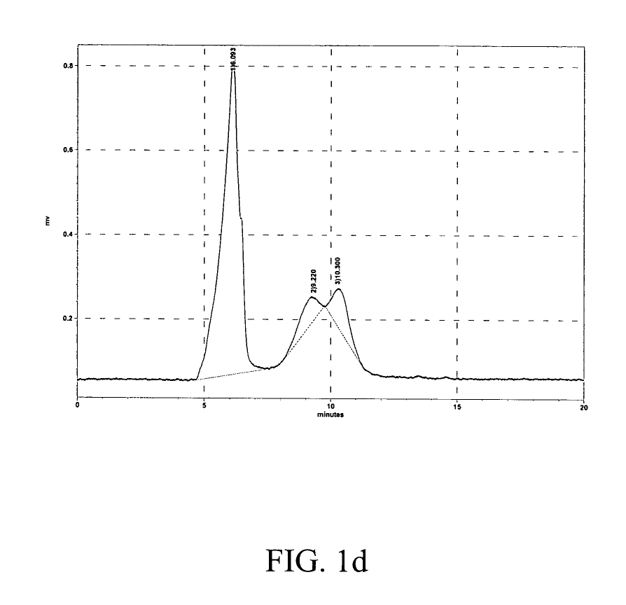

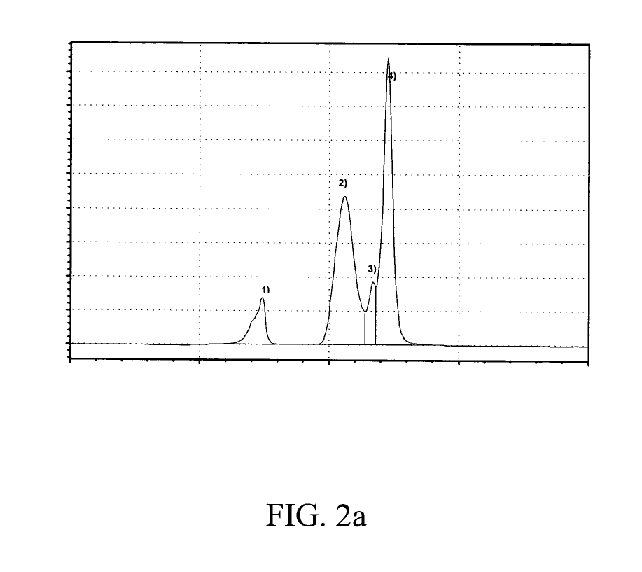

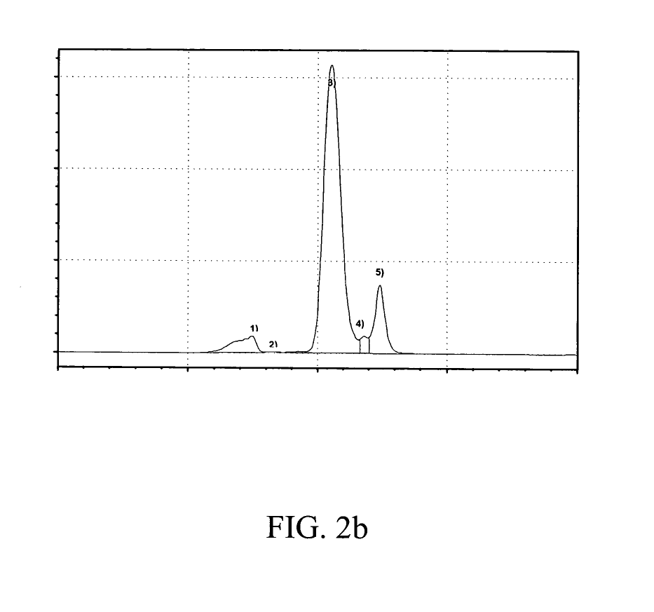

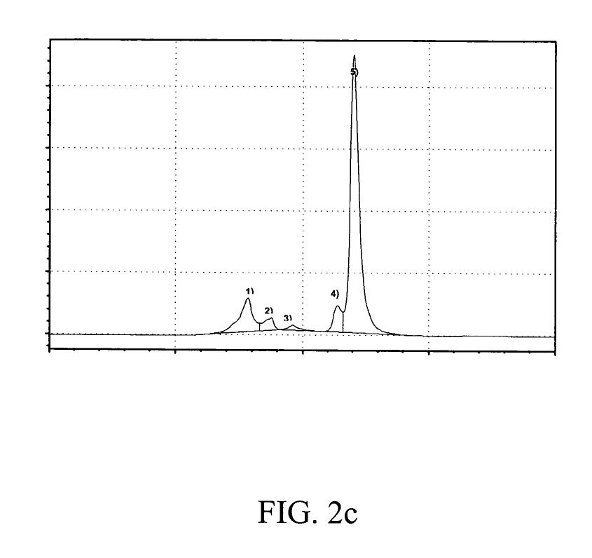

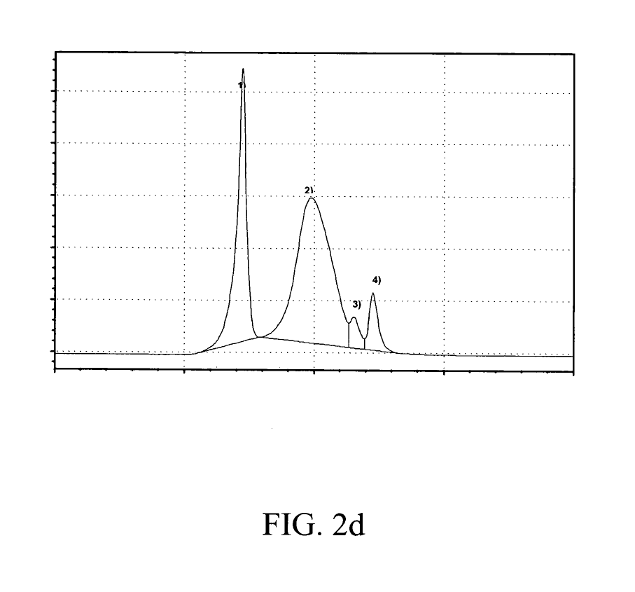

Five liters (L) of the resulting extract mixed with 35 L of distilled water was subjected to a hollow fiber filtration cartridge (0.2 μm, membrane 3.9 m²) with a pump for removing the water-insoluble fraction. Distilled water was added at 50, 61 and 79 min for reducing feeding pressure. The hollow fiber filtration was terminated at 105 min, and 50.4 L of dark yellow solution was obtained. Ten liters (L) of the dark yellow solution was drained into the hollow fiber filtration cartridge (0.2 μm, membrane 3.9 m²) in circulate for removing small molecular weight components. A GPC spectrometer was utilized in monitoring the molecular weight distribution of polysaccharides (as shown in FIG. 2). When the area of the S phase was less than 5% of the total area, the ultra-filtration was completed and the solution was collected. The resulting light yellow powder was obtained after concentrating under reduced pressure.

The preparation of the extract of Astragalus radix was similar to that as mentioned above.

### EXAMPLE 5

### Astragalus Radix Extract and Codonopsis Pilosulae Radix Extract Prepared with Hollow Fiber Filtration Cartridge and Cross-Flow Sluice Cassette

The 50 Kg ground Astragalus radix was placed in a 1500 L tank for heating to 65° C. and stirring with 500 L of distilled water for 2 hours. The resulting opaque suspension was filtered with a #400 filter. The water-insoluble fraction was heated to 65° C. and stirred again with 360 L of distilled water for 2 hours and the insoluble fraction was removed with a #400 filter and centrifugation. The water-soluble fractions from the extractions were collected to provide 815 L of extract for concentrated under reduced pressure at 55° C. The resulting brown extract with a 54.5 Kg weight was obtained.

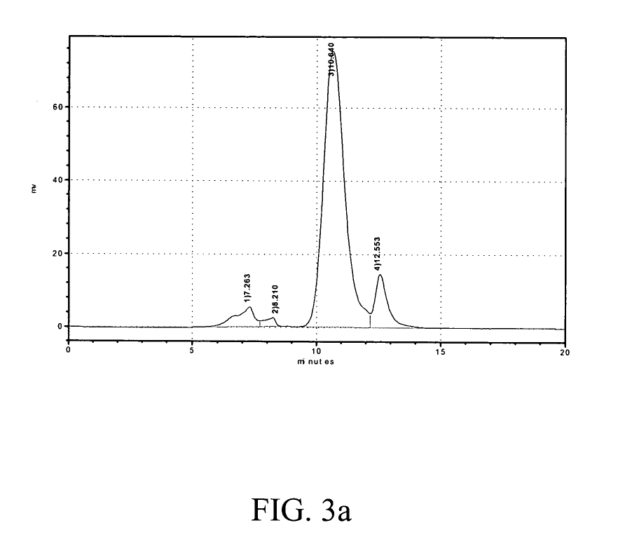

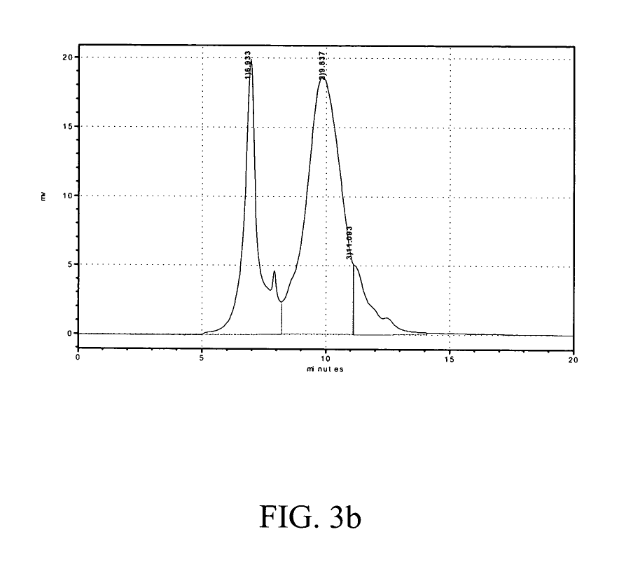

Five liters (L) of the resulting extract and 35 L of distilled water was subjected to a hollow fiber filtration cartridge (0.2 μm, membrane 3.9 m²) with a pump for removing water-insoluble fraction. Five liters (L) of distilled water was added on 22 and 36 min for reducing the feeding pressure. The hollow fiber filtration was terminated on 65 min, and 51.3 L of dark yellow solution was obtained with an average filtration efficiency of 50 L/hr and a yield of 73%. The dark yellow solution 3.3 L was drained into the cross-flow sluice cassette (10 kD, polyethersulfone membrane) in circulate for removing small molecular weight components. A GPC spectrometer was utilized in monitoring the molecular weight distribution of polysaccharides (as shown in FIG. 3). When the area of the S phase was less than 5% of the total area, the ultra-filtration was completed and the solution was collected. The resulting light yellow powder was obtained after concentrating under reduced pressure.

The preparation of the extract of Codonopsis pilosulae was similar to that as mentioned above.

### EXAMPLE 6

### Analysis of Astragalus Radix Extract and Codonopsis Pilosulae Radix Extract Prepared with Hollow Fiber Filtration Cartridge and Cross-Flow Sluice Cassette

Dextran with molecular weights of 670 k, 150 k, 50 k, 35.6 k, 9.9 k, and 1 k and glucose with a molecular weight of 180 were prepared as a standard solution with a concentration of 10 mg/mL. The standard solution was filtered with a 0.2 mm membrane (Millipore®), and the filtrate was subjected to GPC spectrum analysis. The condition of GPC was listed below: (1) column: Shodex™ SUGAR KS-804; (2) flow rate: 1 mL/min; (3) injection volume: 10 μm; and (4) total time: 20 min.

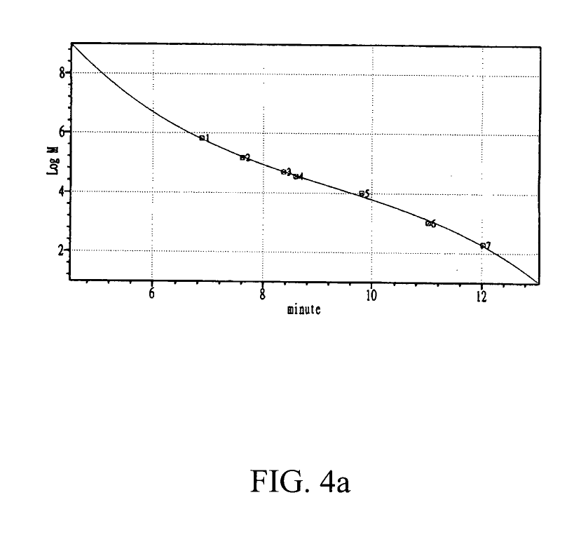

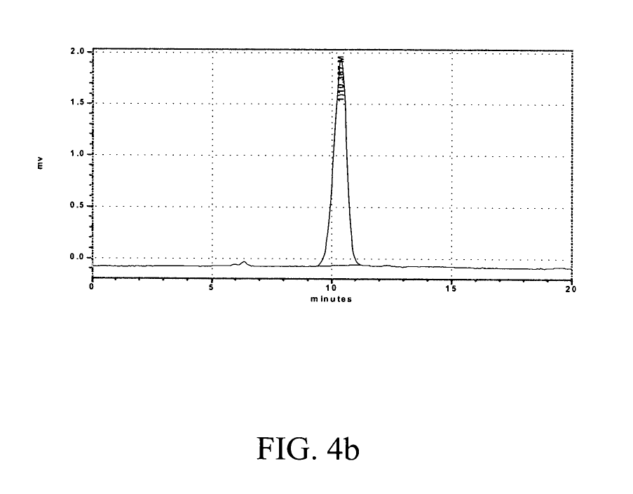

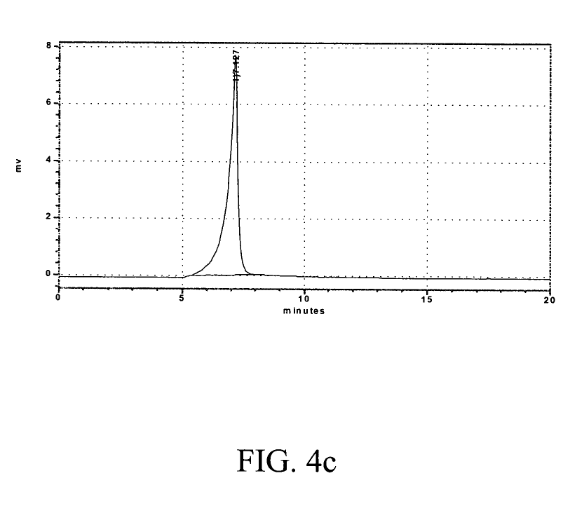

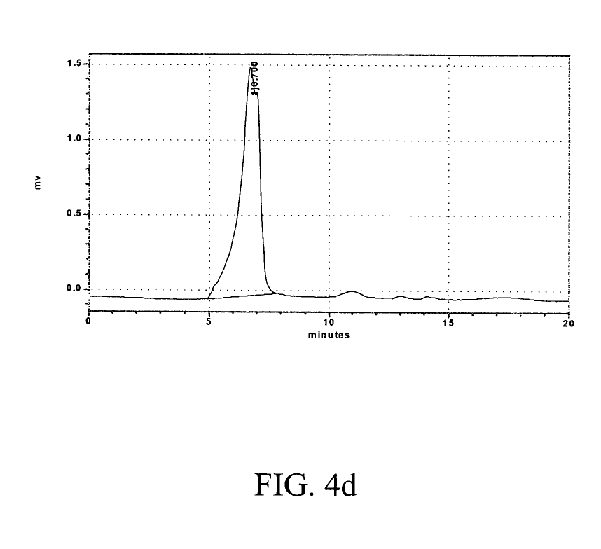

Ten mg of HG>50 k (Astragalus radix), DS-3 k (Codonopsis pilosulae) and DS>50 k (Codonopsis pilosulae) prepared in Example 5 were mixed with 1 mL of distilled water and the resulting solution was filtered with a 0.2 μm membrane (Millipore®). The filtrates were subjected to a GPC spectrum analysis to estimate the molecular weight. The spectrograms of HG>50 k, DS-3 k and DS>50 k was shown in FIG. 4. The peak purity was 100% under integration with conventional software.

According to the regression equation, the ranges of molecular weight of HG>50 k, DS-3 k and DS>50 k were estimated to 125087.3 to 2.06 E+8, 844.981 to 15469.28 and 86453.25 to 1.2 E+8, respectively.

### EXAMPLE 7

### Effects of SG-W, SG-F1, SG-F2 and SG-F3 on Treating Metastasis

SG-W, SG-F1, SG-F2 and SG-F3 were prepared as described in Examples 1 and 2.

Male BALB/c mice aged six (6) weeks were purchased from the National Laboratory Animal Center in Taiwan and raised in light for 12 hours and in dark for 12 hours at a temperature of 22±2° C. Food and water were supplemented sufficiently. The mice were divided into several groups, and each group contained eight (8) mice.

Colon cancer cell line CT-26 were cultured in IMDM medium containing 10% fetal bovine serum (FBS) at 37° C. and 5% CO₂ for implantation. The mice were subjected to intra-splenic implantation with CT-26 (2×10⁴ cells/mouse) when eight (8) weeks old. The mice were weighted and anesthetized by receiving pentobarbital (10 μL/g, 6.5 mg/mL). The 100 μL of CT-26 after concentration adjustment were injected into the mice's spleens and then the mice were sutured with clips. SG-W, SG-F1, SG-F2 and SG-F3 were administrated to the mice. Fourteen days after implantation, the mice were sacrificed, autopsied and the nodules in the livers were observed.

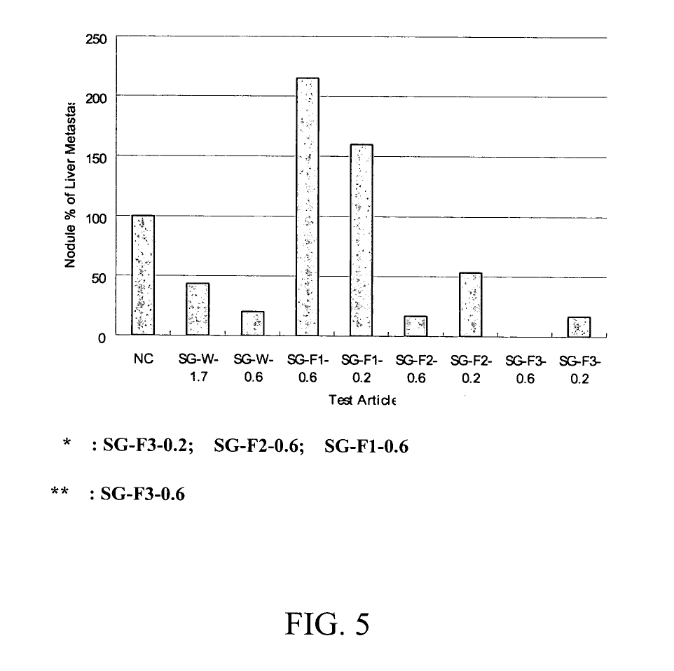

The results were shown in FIG. 5. It represented that administrating SG-F1 of 0.2 and 0.6 g/kg cannot inhibit but enhance metastasis (*p<0.05). SG-F3 of 0.6 g/kg significantly inhibits metastasis (**p<0.01), while SG-F2 of 0.2 and 0.6 g/kg inhibits metastasis to some degree (*p<0.05).

### EXAMPLE 8

### Effective Dosage of SG-F3

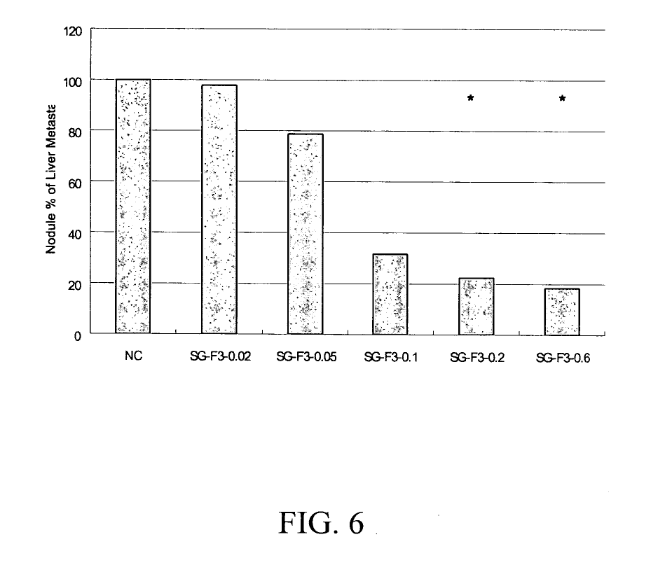

The dosages of SG-F3 used in the example were 0.02 g/kg, 0.05 g/kg, 0.1 g/kg, 0.2 g/kg and 0.6 g/kg. The animal model was similar to that of Example 7. The result is shown in FIG. 6.

It shows that the numbers of nodules in the liver decreases with the dosage increasing. Significantly, dosages of 0.2 g/kg and 0.6 g/kg show good effects on inhibiting metastasis (*p<0.05).

### EXAMPLE 9

### Effects of Astragalus Radix Extract, Codonopsis Pilosulae Radix Extract and Mixed Extract Prepared with Ultra-Filtration on Treating Metastasis

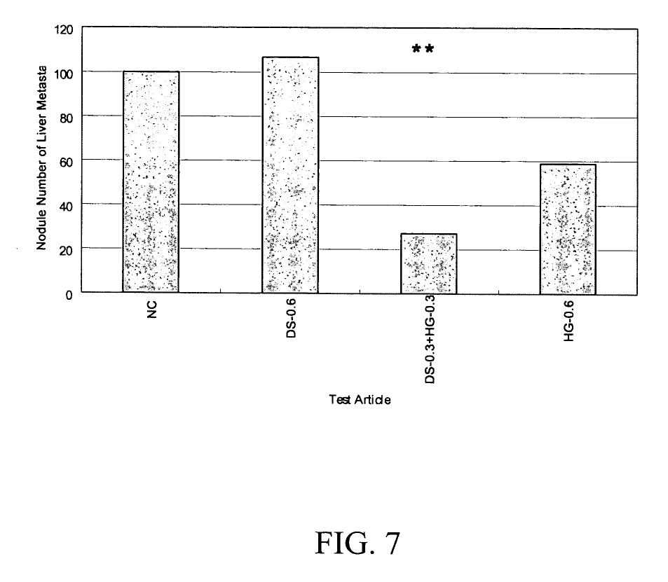

The Astragalus radix extract and Codonopsis pilosulae radix extract were prepared in Example 3. Different ratios of the Astragalus radix extract and Codonopsis pilosulae radix extract were mixed to investigate their effects. The total dosage of Astragalus radix extract and Codonopsis pilosulae radix extract was 0.6 g/kg. The animal model was similar to that of Example 7. The result is shown in FIG. 7.

It shows that the Astragalus radix extract or the Codonopsis pilosulae radix extract only cannot inhibit matastasis. On the other hand, 1:1 ratio of the Astragalus radix and Codonopsis pilosulae radix mixed extract significantly inhibits metastasis (**p<0.01).

### EXAMPLE 10

### Effects of Astragalus Radix Extract and Codonopsis Pilosulae Radix Extract and Mixed Extract Prepared with Hollow Fiber Filtration

Cartridge on Treating Metastasis

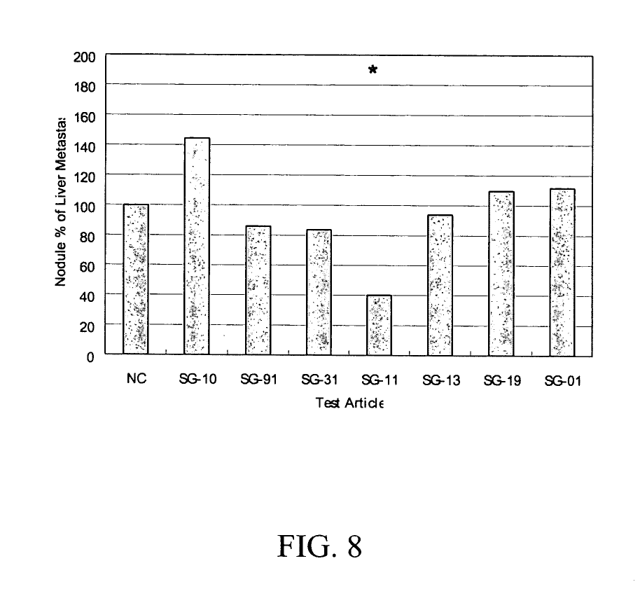

The Astragalus radix extract and Codonopsis pilosulae radix extract were prepared in Example 4. Different ratios of the Astragalus radix extract and Codonopsis pilosulae radix extract were mixed to investigate their effects. The total dosage of Astragalus radix extract and Codonopsis pilosulae radix extract was 0.6 g/kg. The animal model was similar to that of Example 7. The result is shown in FIG. 8.

It shows that the Astragalus radix extract or the Codonopsis pilosulae radix extract only cannot inhibit matastasis. On the other hand, 1:1 ratio of the Astragalus radix and Codonopsis pilosulae radix mixed extract significantly inhibits metastasis (*p<0.05).

### EXAMPLE 11

### Effects of Astragalus Radix Extract and Codonopsis Pilosulae Radix Extract Prepared with Hollow Fiber Filtration Cartridge and Cross-Flow Sluice Cassette on Treating Metastasis

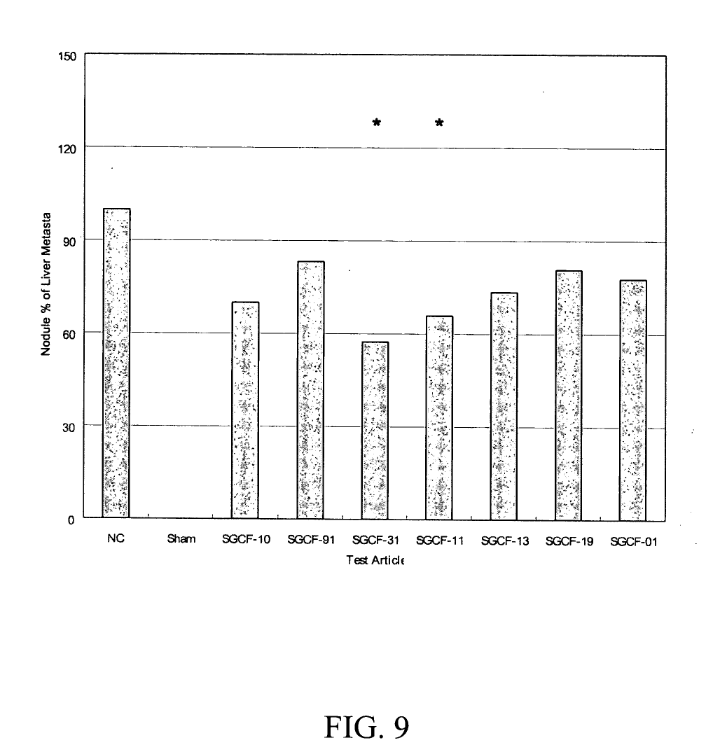

The Astragalus radix extract and Codonopsis pilosulae radix extract were prepared in Example 5. Different ratios of the Astragalus radix extract and Codonopsis pilosulae radix extract were mixed to investigate their effects. The total dosage of Astragalus radix extract and Codonopsis pilosulae radix extract was 0.6 g/kg. The animal model was similar to that of Example 7. The result is shown in FIG. 9.

It shows that the Astragalus radix extract or the Codonopsis pilosulae radix extract only cannot inhibit matastasis. On the other hand, 1:1 and 1:3 ratios of the Astragalus radix and Codonopsis pilosulae radix mixed extracts significantly inhibits metastasis (*p<0.05).

### EXAMPLE 12

### Effects of SG-F3 on Treating Mammary Adenocarcinoma Implanted in Subdermal Area

JC cell line (mammary adenocarcinoma) and culture. JC cell line was a mammary adenocarcinoma cell line which was kindly provided by Dr. Jang, B. L. from the College of Medicine of the National Taiwan University. The cells laminated and attached to the wall of a flask and had a rod and branch shape. The medium for maintenance was RPMI-1640 (Biological Industries®), 10% FBS, 1% sodium pyruvate and 0.5% glucose. The cells were harvested by infiltration with PBS, and then treated with trypsin-EDTA after removing the PBS. After the cells detached the wall of flask slightly, they were resuspended in RPMI medium after removal of trypsin-EDTA. The cells had a survival rate of 95% that was estimated by trypan blue dying.

Implantation and treatment: 5×10⁵ JC cells were used to implant in the subdermal area.

Treatment. SG-F3 as prepared in Example 2 was utilized in the example. Dosages of 0.1 g/kg, 0.3 g/kg and 0.6 g/kg of SG-F3 were administrated to the mice from A31.

Sacrifice and sampling. The animals were sacrificed on A322 according to the CO₂ method. The tumors in the subdermal area were taken and weighted.

Statistic: The data was subjected to p value analysis with SAS program.

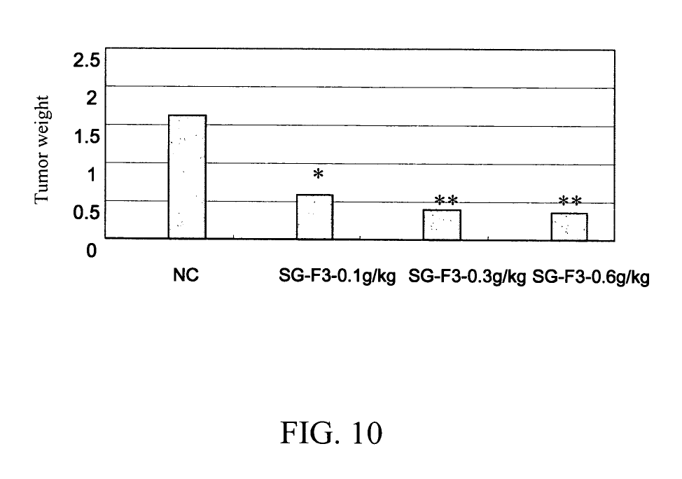

Result. The tumor weights (as shown in FIG. 10) in the animals treated with SG-F3 with three dosages all significantly reduced when compared with the control group (1.62±0.73 g). Furthermore, it also showed that the inhibition effect on tumor growth was in proportion to the dosage, and wherein the high dosage group was evidenced to have the best effect (**p<0.01).

### EXAMPLE 13

### Effect of Astragalus Radix Extract and Codonopsis Pilosulae Radix Extract Prepared with Hollow Fiber Filtration Cartridge and Cross-Flow Sluice Cassette on Treating Mammary Adenocarcinoma Implanted in Subdermal Area

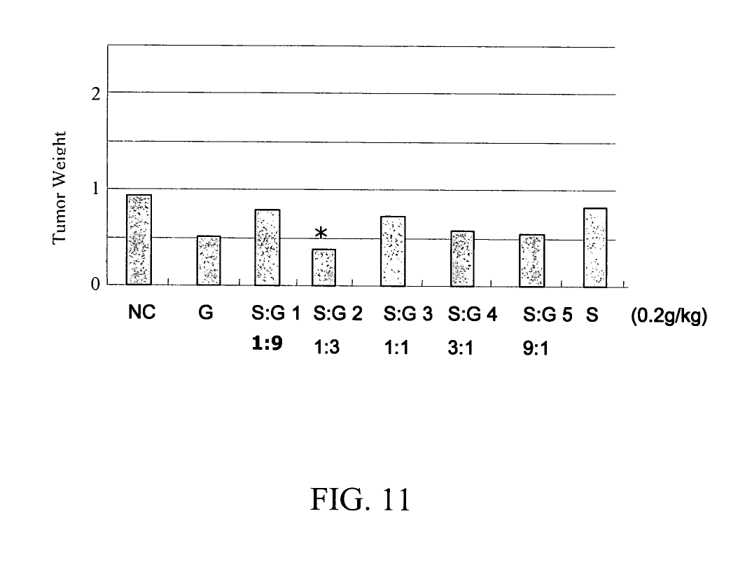

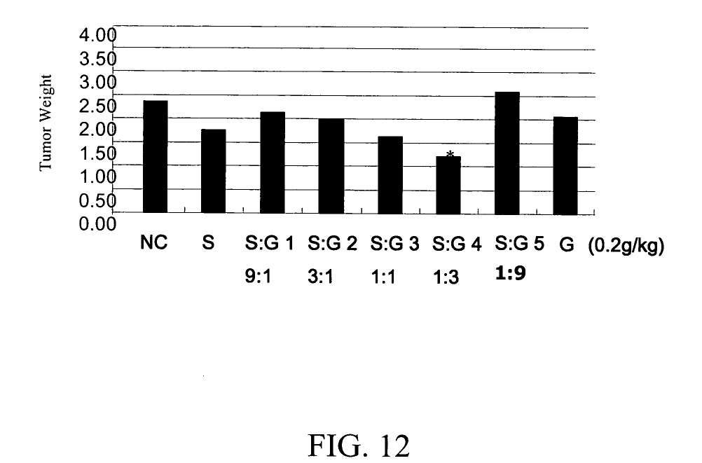

The Astragalus radix extract and Codonopsis pilosulae radix extract were prepared in Example 5. Different ratios of the Astragalus radix extract and Codonopsis pilosulae radix extract were mixed to investigate their effects. The total dosage of the Astragalus radix extract and Codonopsis pilosulae radix extract were 0.2 g/kg. The animal model was similar to that of Example 12 except that the treatment began on D7 and the sacrifice was carried on D34. The result is shown in FIG. 11.

It shows that 3:1 and 9:1 ratios of the Astragalus radix and Codonopsis pilosulae radix mixed extracts significantly inhibit tumor growth (*p<0.05).

### EXAMPLE 14

### Extracts of Astragalus Radix Extract and Codonopsis Pilosulae Radix Extract Prepared with Hollow Fiber Filtration Cartridge and Cross-Flow Sluice Cassette on Treating Lung Carcinoma Implanted in Subdermal Area

LL/2 cell line (lung carcinoma) and culture: LL/2 cell line was a Lewis lung carcinoma cell line of C57BL mice which was purchased from the Food Industry Research and Development Institute (FIRDI), Hsinchu, Taiwan. The cells clustered and slightly attached to the wall of a flask. The medium for maintenance was MEM (Gibco®) and 10% FBS (Biological Industries®) at 37° C. and 5% CO₂. The cells were harvested by filtration with PBS, and then treated with trypsin-EDTA after removing the PBS. After the cells detached the wall of flask slightly, they were resuspended in RPMI medium after removal of trypsin-EDTA. The cells had a survival rate of 95% that was estimated by trypan blue dying.

Implantation and treatment: 5×10⁵ LL/2 cells were used to implant in the subdermal area.

Treatment: The Astragalus radix extract and Codonopsis pilosulae radix extract as prepared in Example 5 was utilized in the example. Different ratios of the Astragalus radix extract and Codonopsis pilosulae radix extract were mixed to investigate their effects. The total dosage of the Astragalus radix extract and Codonopsis pilosulae radix extract were 0.2 g/kg and treated from D8.

Sacrifice and sampling. The animals were sacrificed on D22 according to the CO₂ method. The tumors in the subdermal area were taken and weighted.

Statistic. The data was subjected to p value analysis with SAS program.

Result. It shows that the tumor weight of the animals treated with a 1:1 ratio of the Astragalus radix extract and Codonopsis pilosulae radix extract were significantly reduced by 31%. In addition, the tumor weight of 3:1 ratio was the lightest in the experimental groups, which was reduced by 49% (*p<0.05).

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

1. A composition for inhibiting carcinogenesis and metastasis, comprising a therapeutically effective amount of an Astragalus radix and Codonopsis pilosulae radix mixed extract.

2. The composition according to claim 1, wherein the weight ratio of Astragalus radix:Codonopsis pilosulae radix in the mixed extract is from 3:1 to 1:3.

3. The composition according to claim 1, wherein Astragalus radix is Astragalus mogholicus or Astragalus membranaceus radix.

4. The composition according to claim 1, wherein Codonopsis pilosulae radix is Codonopsis pilola (Franch.) Nannf, Codonopsis tangshen Oliv. or Codonopsis pilola (Franch.) var. modesta (Nannf.) L. T. Shen radix.

5. The composition according to claim 1, wherein the composition is for inhibiting carcinogenesis and metastasis of colon cancer, lung carcinoma or mammary adenocarcinoma.

6. The composition according to claim 1, which is in the form of tablet, capsule, solution, tonic, or food.

7. A method of producing the Astragalus radix and Codonopsis pilosulae radix mixed extract according to claim 1, comprising (A) co-extracting Astragalus radix and Codonopsis pilosulae radix with water.

8. The method according to claim 7, wherein the weight ratio of Astragalus radix:Codonopsis pilosulae radix is from 3:1 to 1:3.

9. The method according to claim 7, wherein the method comprises:
(A11) co-extracting Astragalus radix and Codonopsis pilosulae radix with water at a temperature of from 45 to 95° C. to separate a water-insoluble fraction from a water-soluble fraction; and
(A12) concentrating the water-soluble fraction at a temperature of from 40 to 80° C. and at a pressure of from 0 to 100 mmHg.

10. A method of producing the Astragalus radix and Codonopsis pilosulae radix mixed extract according to claim 1, comprising (B) co-extracting Astragalus radix and Codonopsis pilosulae radix with water and ethanol.

11. The method according to claim 10, wherein the weight ratio of Astragalus radix:Codonopsis pilosulae radix is from 3:1 to 1:3.

12. The method according to claim 10, wherein the method comprises:
(B1) co-extracting Astragalus radix and Codonopsis pilosulae radix with 90 to 99% ethanol to separate an ethanol-insoluble fraction from an ethanol-soluble fraction;
(B2) extracting the ethanol-insoluble fraction from step (B1) with water at a temperature of from 45 to 95° C. and obtaining a water-soluble fraction; and
(B3) combining the ethanol-soluble fraction from step (B1) and the water-soluble fraction from step (B2) and concentrating at a temperature of from 40 to 80° C. and at a pressure of from 0 to 100 mmHg.

13. The method according to claim 7, comprising:
(A21) extracting Astragalus radix and Codonopsis pilosulae radix with water, respectively, to separate a water-insoluble fraction from a water-soluble fraction;
(A22) ultra-filtrating the water-soluble fraction from step (A21) with an ultrafilter having a 5 to 10 kD molecular weight cutoff;
(A23) concentrating the filtrates from step (A22); and
(A24) combining and mixing filtrates of Astragalus radix and Codonopsis pilosulae radix from step (A23) to yield the Astragalus radix and Codonopsis pilosulae radix mixed extract.

14. The method according to claim 13, wherein the weight ratio of Astragalus radix:Codonopsis pilosulae radix of step (A21) is from 3:1 to 1:3.

15. The method according to claim 13, wherein extracting Astragalus radix and Codonopsis pilosulae radix with water in step (A21) is performed at a temperature of from 45 to 95° C.

16. The method according to claim 13, wherein ultra-filtrating the water-soluble fraction of Astragalus radix and Codonopsis pilosulae radix in step (A22) is with an ultrafilter having a 5 kD molecular weight cutoff and then with an ultrafilter having a 10 kD molecular weight cutoff.

17. The method according to claim 13, wherein concentrating the filtrates from step (A22) in step (A23) is performed at a temperature of from 40 to 80° C. and at a pressure of from 0 to 100 mmHg.

18. The method according to claim 7, comprising:
(A31) extracting Astragalus radix and Codonopsis pilosulae radix with water, respectively, to separate a water-insoluble fraction from a water-soluble fraction;
(A32) concentrating the water-soluble fractions from step (A31) at a temperature of from 40 to 80° C. and at a pressure of from 0 to 100 mmHg;
(A33) ultra-filtrating and ultra-dialying the concentrated water-soluble fraction from step (A32) with an ultrafilter having a 1 to 4000 kD molecular weight cutoff;
(A34) mixing the filtrates from step (A33) to yield the Astragalus radix and Codonopsis pilosulae radix mixed extract.

19. The method according to claim 18, wherein the weight ratio of Astragalus radix:Codonopsis pilosulae radix of step (A31) is from 3:1 to 1:3.

20. The method according to claim 18, wherein ultra-filtrating the concentrated water-soluble fraction from step (A32) of step (A33) is performed with a hollow fiber filtration cartridge and/or a cross-flow sluice cassette.
